(19) United States
(12) Patent Application Publication
(10) Pub. No.: US 2021/0307638 A1
(43) Pub. Date: Oct. 7, 2021

(54) SYSTEM, METHOD AND ACCESSORIES FOR DIELECTRIC-BASED IMAGING

(71) Applicant: Navix International Limited, Road Town (VG)

(72) Inventor: Shlomo BEN-HAIM, Milan (IT)

(73) Assignee: Navix International Limited, Road Town (VG)

(21) Appl. No.: 17/352,396

(22) Filed: Jun. 21, 2021

Related U.S. Application Data
(63) Continuation of application No. PCT/EP2019/086868, filed on Dec. 20, 2019.
(60) Provisional application No. 62/788,969, filed on Jan. 7, 2019.
(60) Provisional application No. 62/782,562, filed on Dec. 20, 2018.

(51) Int. Cl. A61B 5/0536; A61B 5/0538; A61M 25/09

(52) U.S. Cl. CPC A61B 5/0536; A61M 25/09; A61B 5/0538

(57) ABSTRACT

A method of performing dielectric-based imaging is disclosed comprising exciting at least one pair of electrodes according to an excitation scheme, the at least one pair of electrodes comprising at least one pair of in-body electrodes (also referred herein below intra-body electrode) located inside of the examined living body, measuring and recording voltages developing on the in-body electrodes during the excitation according to the excitation scheme, solving an inverse problem to achieve a 3D dielectric map from the recorded voltages and optionally providing a 3D image of the body tissues based on the 3D dielectric map.

### RELATED APPLICATIONS

This application is a Continuation of PCT Patent Application No. PCT/EP2019/086868 having International filing date of Dec. 20, 2019, which claims the benefit of priority of U.S. Provisional Patent Applications Nos. 62/782,562 filed on Dec. 20, 2018 and 62/788,969 filed on Jan. 7, 2019. The contents of the above applications are all incorporated by reference as if fully set forth herein in their entirety.

### FIELD AND BACKGROUND OF THE INVENTION

The present invention, in some embodiments thereof, relates to medical imaging and, more specifically, but not exclusively, to systems and methods for conductivity based imaging, e.g., for reconstruction of body tissues and organs.

Electrical Impedance Tomography (EIT) system and method of medical imaging, as is known in the art, is implemented by deploying electrodes at the body's surface of a subject, injecting electrical excitation to some of the employed electrodes, measuring the electrical signals received at the other employed electrodes, calculating, based on the measured signals, 3D image(s) of tissues and organs inside the body and providing display of the calculated 3D images.

The 3D imaging is based on the fact that muscle and blood conduct the applied currents better than fat, bone, or lung tissue. However, the 3D imaging that is based on reconstructed conductivity has found only limited utility, because of the low resolution of the obtained images.

There is a need for system and method that provide accurate imaging of body organs and lumens, which do not employ ionized radiation or which minimize ionized radiation.

### SUMMARY OF THE INVENTION

The present invention, in some embodiments thereof, relates to medical imaging and, more specifically, but not exclusively, to systems and methods for dielectric based imaging, e.g., for reconstruction of body tissues and organs. Dielectric based imaging may also be referred herein as conductivity based imaging.

A method of performing dielectric-based imaging is disclosed comprising exciting at least one pair of electrodes according to an excitation scheme, the at least one pair of electrodes comprising at least one pair of inner electrodes (also referred herein below as intra-body electrode or in-body electrode, or in tissue electrode) located inside of an examined living tissue or body, measuring and recording voltages developing on the in-body electrodes during the excitation according to the excitation scheme, solving an inverse problem to achieve a 3D dielectric map from the recorded voltages and optionally providing a 3D image of the body tissues based on the 3D dielectric map. In some embodiments, the at least one pair of in-body electrode include 6, 7, 8, 10, 12, 14, 16, 18 or 20 pairs of electrodes.

According to some embodiments of the present invention there is provided a method of performing dielectric-based imaging. The method comprising: receiving voltage measurements developed on at least one pair of in-body electrode located inside of the examined body during an excitation according to an excitation scheme; wherein the excitation scheme includes exciting one or more electrodes of the at least one pair of in-body electrode; solving an inverse problem to achieve a 3D dielectric map from the measured voltages; and providing a 3D image of the body tissues based on the 3D dielectric map.

The at least one pair of in-body electrodes is provided on or embodied in a catheter or a guidewire. The catheter may include or may be: micro catheter with electrodes, sheath with electrodes, spiral catheter with electrodes, basket catheter with electrodes, pig tail catheter with electrodes. As would be understood by the skilled person, a guidewire is a thin, medical wire that is flexible or has a flexible portion that is configured to be inserted into a body. A guidewire is configured to guide another medical device, such as a catheter, central venous line, or feeding tube inside the body. Reference to guiding a medical device using a guidewire means to assist in inserting, positioning and moving such a medical device in the body. Guidewires may be used with any other suitable medical device that is to be guided in a body. A guidewire may have a diameter less than 5 mm, optionally 1 mm, optionally less than 0.5 mm, and may have a length between 10 cm and 10 m, optionally between 50 cm and 5 m, optionally between 80 cm and 450 cm, optionally between 100 cm and 250 cm.

Where reference is made herein to a catheter, it would be understood that the term catheter refers to an elongate, flexible implement, for example a tube or comprising a tube, suitable for insertion into a living body, the catheter, for example the tube, carrying one or more electrodes. The catheter may further be suitable to be withdrawn from the body following insertion of the catheter into the body, for example following a medical procedure. The tube may be flexible or may have at least a flexible portion and may have varying levels of stiffness depending on the medical procedure(s) that the catheter is configured to carry out. The catheter may have a rigid portion and a flexible portion. In some examples, the catheter is a tube with a diameter of between 0.3 mm and 0.9 mm. In some examples, the catheter is a tube and further comprises a sheath surrounding the tube. In other words, the catheter comprises an outer tube (the sheath) within which is an inner tube. The sheath may be retractable, i.e. the inner tube may be moveable along a longitudinal direction relative to the sheath. In these examples, one or more electrodes may be disposed on the sheath or the inner tube, or one or more electrodes may be disposed on each of the sheath and the inner tube. The tube, inner tube or outer tube may be made of any suitable respective material, for example extruded polymer material, a mesh or braid of any suitable material, and the like.

In some examples, a catheter may be configured to allow the insertion or withdrawal of fluids from the body. In some examples a catheter may be configured to distend body passages, and in some examples, a catheter may be configured to deliver a treatment to a portion of the body. In some examples, catheters are configured to be inserted into the body via a blood vessel, such as an artery or a vein. In one specific example, the catheter is configured to be inserted into a heart chamber, for example via the femoral artery.

In embodiments, the catheter may be a basket catheter, such as the basket catheter depicted in FIG. 12. That is, in these examples, the catheter may be a tube and may further comprise a basket portion (such as basket portion 124C depicted in FIG. 12) at an end of the tube. The basket portion may comprise a plurality of strands 126C. Each strand 126C may include a plurality electrodes 128C, optionally arranged in pairs. In other words, in these embodiments, the electrodes carried by the catheter may be carried by the basket portion as well as or instead of the tube portion of the catheter.

In some embodiments, the medical device may include or may be: a scissors, scalpel, knife, cannula, clamp, needle, syringe, laparoscope, guidewire, suture thread with electrodes or another tool having a designated medical use. The medical device may be carrying one or more electrode pairs for insertion the one or more electrodes into a living body, for dielectric-based imaging. In some embodiments, the medical device comprises a working portion used for performing a tissue manipulation (e.g., a blade, sharpened tip, and/or tissue gripping portion) and a non-working portion. In some embodiments, the electrodes may be attached to or make part of the non-working portion, the working portion, or both.

According to some embodiments, the excitation is applied to the at least one pair of intra-body electrodes located inside of the examined body. In some embodiments, a plurality of intra-body electrodes may be provided, optionally arranged in pairs. In some embodiments, excitation may include injecting current between pair of electrodes, when one of the electrode pairs is grounded.

A method of performing conductivity-based imaging is disclosed comprising exciting at least one pair of electrodes according to an excitation scheme, the at least one pair of electrodes comprising a surface electrode located on the surface of an examined living body and at least one in-body electrode (also referred herein below intra-body electrode) located inside of the examined living body, measuring and recording voltages developing on the surface electrodes and on the in-body electrodes during the excitation according to the excitation scheme, solving an inverse problem to achieve a 3D conductivity map from the recorded voltages and optionally providing a 3D image of the body tissues based on the 3D conductivity map.

A method of providing 3D conductivity map of body tissues is disclosed comprising exciting at least one pair of electrodes according to an excitation scheme, the at least one pair of electrodes comprising a surface electrode located on the surface of an examined living body and at least one in-body electrode located inside of the examined living body, measuring voltages developing on the surface electrodes and on the in-body electrodes during the excitation according to the excitation scheme, solving an inverse problem to obtain a 3D conductivity map from the measured voltages. The method may comprise providing (e.g., displaying) a 3D image of the body tissues based on the 3D conductivity map.

In some embodiments the excitation is applied to at least one additional pair of electrodes that comprises a surface electrode located on the surface of an examined body and at least one in-body electrode located inside of the examined living body.

In some embodiments the steps of exciting and measuring are repeated M times, while the in-body electrodes are moved between cycles of excitation.

In some embodiments a step of averaging and weighing the measurements of the step of measuring is performed before the step of solving.

A method of imaging body tissues is disclosed comprising exciting at least one pair of electrodes according to an excitation scheme, the at least one pair of electrodes comprising a surface electrode located on the surface of an examined living body and at least one in-body electrode located inside of the examined living body, measuring voltages developing on the surface electrodes and on the in-body electrodes during the excitation according to the excitation scheme, solving an inverse problem to obtain a 3D conductivity map from the measured voltages. The method may comprise providing (e.g., displaying) a 3D image of the body tissues based on the 3D conductivity map.

A method of imaging a volume in an examined living body is disclosed comprising exciting at least one pair of electrodes according to an excitation scheme, the at least one pair of electrodes comprising a surface electrode located on the surface of an examined living body and at least one in-body electrode located inside of the examined living body, measuring voltages developing on the surface electrodes and on the in-body electrodes during the excitation according to the excitation scheme, solving an inverse problem to obtain a 3D conductivity map from the measured voltages. The method may comprise providing (e.g., displaying) a 3D image of the volume based on the 3D conductivity map.

According to some embodiments of the present invention there is provided a method of performing conductivity-based imaging, the method comprising: exciting at least one pair of electrodes according to an excitation scheme, the at least one pair of electrodes comprising a surface electrode located on the surface of an examined body and at least one in-body electrode located inside of the examined body; measuring and recording voltages developing on the surface electrode and on the in-body electrode during the excitation according to the excitation scheme; solving an inverse problem to achieve a 3D conductivity map from the recorded voltages; and providing a 3D image of the body tissues based on the 3D conductivity map.

According to some embodiments, the excitation is applied to at least one additional pair of electrodes that comprises a surface electrode located on the surface of the examined body and at least one in-body electrode located inside of the examined body.

According to some embodiments, the excitation is applied to at least one additional pair of electrodes that comprises two in-body electrodes.

According to some embodiments, the excitation is applied to at least one additional pair of electrodes that comprises two surface electrodes.

According to some embodiments, the steps of exciting and measuring are repeated a defined number of times (M) with the in-body electrodes at different locations inside the body, wherein M is at least two.

According to some embodiments, the steps of exciting and measuring are repeated at a rate of between 10 and 500 times per second.

According to some embodiments, the method further comprising a step of combining measurements obtained when the in-body electrodes were at different locations to a single set of measurements, and wherein the inverse problem is solved for that single set of measurements.

According to some embodiments, the different locations include at least two locations, each in the vicinity of a different structural feature to be imaged within a volume of the examined body.

According to some embodiments, the solving is executed for each of the M measurements separately, and the obtained solutions are averaged to provide the 3D image.

According to some embodiments of the present invention there is provided a method of providing a 3D image of the body tissues. The method comprising: exciting at least one pair of electrodes according to an excitation scheme, the at least one pair of electrodes comprising a surface electrode located on the surface of an examined body and at least one in-body electrode located inside of the examined body; measuring voltages developing on the surface electrode and on the in-body electrode during the excitation according to the excitation scheme; solving an inverse problem to achieve a 3D conductivity map from the measured voltages; and providing a 3D image of the body tissues based on the 3D conductivity map.

According to some embodiments, the excitation is applied to at least one additional pair of electrodes that comprises a surface electrode located on the surface of an examined body and at least one in-body electrode located inside of the examined body.

According to some embodiments, the excitation is applied to at least one additional pair of electrodes that comprises two in-body electrodes.

According to some embodiments, the excitation is applied to at least one additional pair of electrodes that comprises two surface electrodes.

According to some embodiments, the steps of exciting and measuring are repeated a defined number of times (M) with the in-body electrodes at different locations inside the body, wherein M is at least two.

According to some embodiments of the present invention there is provided a method of obtaining a 3D conductivity map of body tissues. The method comprising: exciting at least one pair of electrodes according to an excitation scheme, the at least one pair of electrodes comprising a surface electrode located on the surface of an examined body and at least one in-body electrode located inside of the examined body; measuring voltages developing on the surface electrode and on the in-body electrode during the excitation according to the excitation scheme; and solving an inverse problem to obtain a 3D conductivity map from the measured voltages.

According to some embodiments of the present invention there is provided a method of performing conductivity-based imaging. The method comprising: receiving voltage measurements developed on a surface electrode located on the surface of an examined body and on an in-body electrode located inside of the examined body during an excitation according to an excitation scheme; wherein the excitation scheme includes exciting the surface electrode and the in-body electrode; solving an inverse problem to achieve a 3D conductivity map from the measured voltages; and providing a 3D image of the body tissues based on the 3D conductivity map.

According to some embodiments of the present invention there is provided a system for performing conductivity-based imaging comprising a controller configured to perform the methods described above when executing executable code stored in its memory.

According to some embodiments of the present invention there is provided a system for performing conductivity-based imaging. The system comprises: a control unit; surface electrodes unit, comprising at least 2 electrodes; intra-body electrodes, comprising at least 2 electrodes; a first communication channel to provide communication between the control unit and the surface electrodes unit; and a second communication channel to provide communication between the control unit and the intra-body electrodes unit.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically depicts deployment of a set of electrodes on and in a body, according to embodiments of the present invention;

FIG. 2 is a schematic illustration of catheter 208, according to embodiments of the present invention;

FIG. 3 schematically depicts electrical field generator/measurer, according to embodiments of the present invention;

FIG. 4 is a schematic block diagram of a system for conductivity-based imaging, according to embodiments of the present invention;

FIG. 5 is a top-level flow of a process for converting a collection of measured voltages on a set of electrodes into a 3D image, according to embodiments of the present invention;

FIG. 6A is a flow chart depicting method for conductivity-based imaging according to embodiments of the present invention;

FIG. 6B is a flow chart depicting method for performing multiple cycles of measurements, according to the invention;

FIG. 7 is a flow chart depicting a method of performing conductivity-based imaging according to some embodiments of the present invention;

FIG. 8A is simulation results of 3D conductivity values reconstructed from voltage measurements made by surface electrodes and in-body electrodes according to some embodiments of the invention;

FIG. 8B shows the actual structure the reconstruction of which is depicted in FIG. 8A;

FIG. 9A is a diagrammatic representation of a general view of a needle according to some embodiments of the invention;

FIG. 9B is a diagrammatic representation of a side view of needle as shown generally in FIG. 9A;

FIG. 9C is a diagrammatic representation of a tip of a needle as shown generally in FIG. 9A;

FIG. 9D is a diagrammatic representation of a top view of a needle as shown generally in FIG. 9A.

FIG. 10 is a diagrammatic representation of a thread 100A, which may be used for dielectric-based imaging, according to some embodiments of the invention;

FIG. 11 is a diagrammatic representation of a flex tape thread 100B, which may be used for dielectric-based imaging, according to some embodiments of the invention;

FIG. 12 is a diagrammatic presentation of a basket catheter 100C according to some embodiments of the invention; and

FIG. 13 is a diagrammatic presentation of a guidewire according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

The present invention, in some embodiments thereof, relates to medical imaging and, more specifically, but not exclusively, to systems and methods for conductivity based imaging, e.g., for reconstruction of body tissues and organs.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. The terms ‘injecting signal’, injecting current’, ‘exciting signal’ and ‘exciting current’ will be all used herein after to describe signals provided to electrodes used in the process of imaging as described below.

The present invention may be a system, a method, and/or a computer program product. The computer program product may include a computer readable storage medium (or media) having computer readable program instructions thereon for causing a processor to carry out aspects of the present invention.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In the following detailed description, the terms catheter refers to tube suitable for insertion into a body, the tube comprising or carrying one or more electrodes and being flexible or having at least a flexible portion. In some examples, the catheter is a tube with a diameter of between 0.3 mm and 0.9 mm. For example, the catheter may be: endoscope, enteral feeding tube, stent, graft, or any other type of flexible tubular medical device carrying one or more electrodes and suitable for insertion into the body.

The catheter may include or may be: micro catheter with electrodes, sheath with electrode, spiral catheter with electrode, basket catheter with electrodes or Pig tail catheter with electrodes.

Systems and methods for intra-body-electrode aided conductivity-based imaging may employ one or more surface electrodes deployed on the surface of an examined body preferably around the examined body organ and one or more intra-body electrodes. Systems and methods for intra-body-electrode aided dielectric-based imaging may employ solely one or more pairs of intra-body electrodes (the one or more surface electrodes may be omitted for performing the dielectric-based imaging). According to embodiments of the present invention, one or more electrodes may be inserted into a living body, carried by or being part of an existing medical device, such as a catheter or a guide wire endoscope, enteral feeding tube, stent, graft, etc. In some embodiments, the medical device may include or may be: a scissors, scalpel, knife, cannula, clamp, needle, syringe, laparoscope, guidewire, suture thread with electrodes or another tool having a designated medical use, carrying one or more electrodes for insertion the one or more electrodes into a living body.

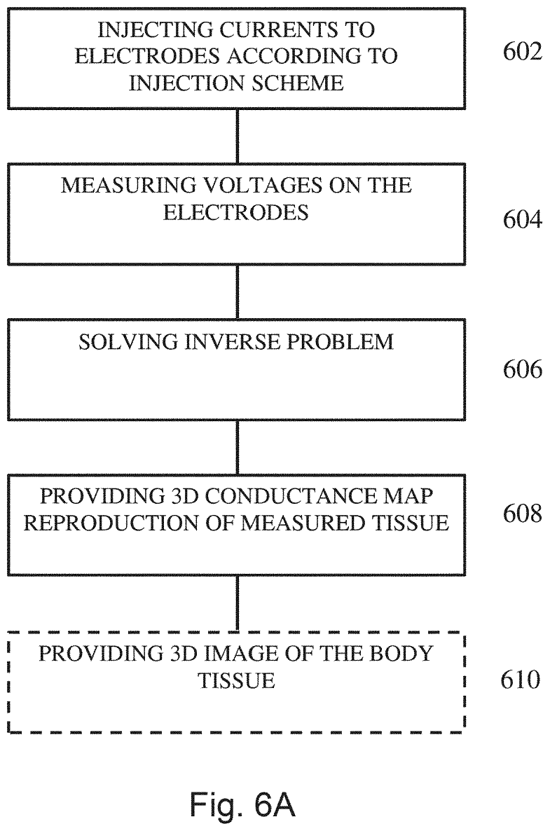

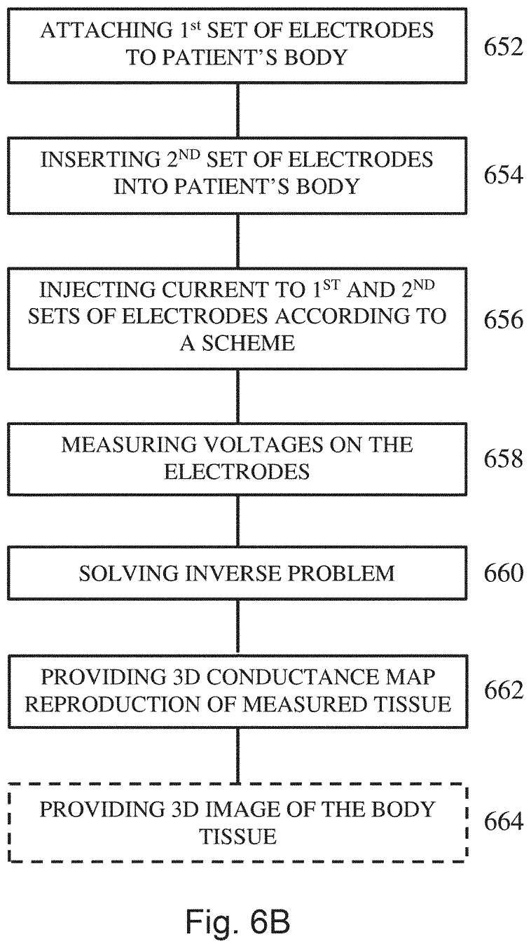

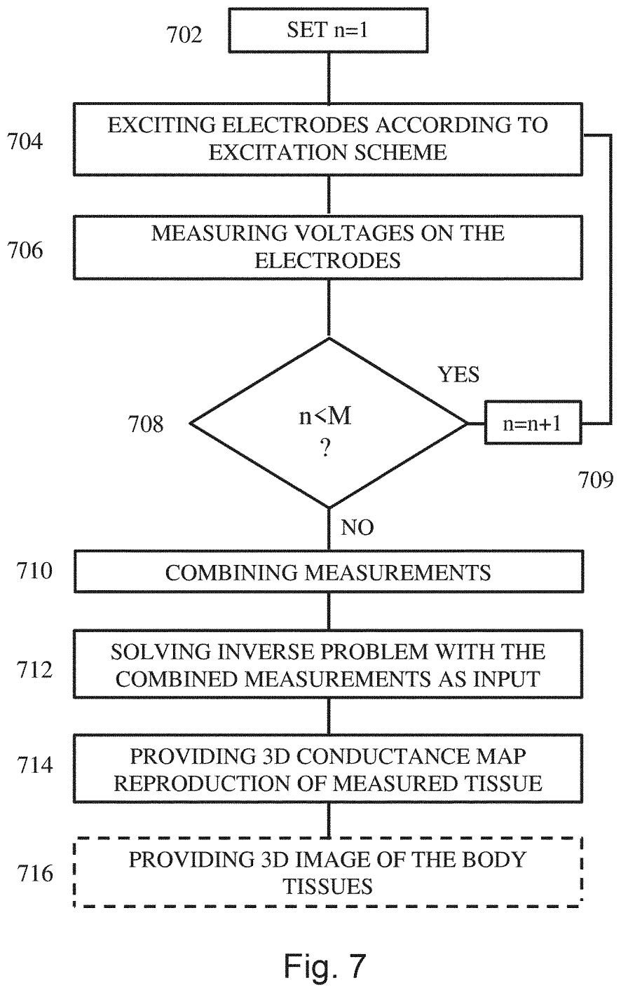

According to some embodiments the insertion of electrodes may be made as part of, or in addition to another medical procedure, e.g. for saving the patient the inconvenience involved in undergoing the physical process more than once. In such instances, according to some embodiments electrical signals that need to be injected to some of the electrodes, and the respective signals that need to be measured on other electrodes of the system, may take advantage of another medical procedure that involves injection and measuring of signals between electrodes (e.g. where such signaling is employed for providing location information of the catheter or other intra-body device used as an electrode carrier) in the body, and may use the signaling process (injection and measuring) for the purpose of intra-body-electrode aided conductivity-based imaging, as is explained in detail below. For example steps 602 and 604 of FIG. 6A or steps 654 and 656 of FIG. 6B or steps 704 and 706 of FIG. 7 may be part of a medical procedures.

Exemplary medical procedures that a catheter may be configured to carry out include catheterization procedures, e.g., cardiac ablation, intestinal ablation, stent or graft deployment, etc. Other exemplary medical procedures that a catheter may be configured to carry out include internal cavity mapping (such as heart chamber mapping for example), and endocardiac ECG recording, and deployment of various different types of implements into the body, such as an annuloplasty ring, a heart valve clip, a left atrial appendage occluder, a ventricular septal defect clip, or other types of heart implants such as heart valve implants. In these examples the catheter is configured to be inserted into the body to carry out the procedure and them subsequently withdrawn from (i.e. taken out of) the body after the procedure. For example, after an implement has been deployed in the body using the catheter, the catheter may be taken out of the body. In some embodiments, intra-body-electrode aided conductivity-based imaging may take advantage of the catheter which is already inserted into the living body and the surface electrode provided as part of such medical procedures. For example, imaging of organs (other organs or in addition to the heart —for example: the aorta and/or the esophagus or the entire chest environment) may be performed using intra-body-electrode aided conductivity-based imaging to a patient undergoing a cardiac ablation procedures.

In some embodiments, intra-body-electrode aided conductivity-based imaging may be implemented to existing system(s) by programming such system to use information already received by such system (e.g., for navigation) to obtain imaging of an organ being treated by such system or other organs. For example, in cardiac ablation procedures: catheter(s) carrying one or more intra-body electrodes and surface electrodes may be used in existing procedures to map the inside of a heart chamber, and the intra-body-electrode aided conductivity-based imaging may supply imaging of the entire chest environment, for example, to show the aorta and/or the esophagus.

Damaging the esophagus is one of the more common complications of left atrium ablation, because the esophagus neighbors the left atrium, and is usually dynamic. Esophagus imaging using intra-body-electrode aided conductivity-based imaging may be used in such ablation procedures, optionally taking advantage of information (e.g., signals) already obtained in such procedures.

The following detailed description is in reference to voltage measurements, it should be noted that embodiments of the present invention are not limited to voltage measurements and may deploy other measurements, such as current and/or impedance measurements. Impedance measurements may be obtained from voltage and current measurements on the one or more electrodes.

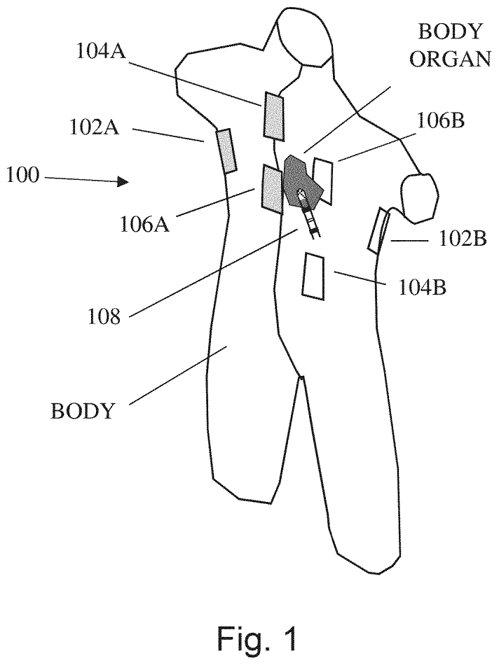

Reference is made to FIG. 1, which schematically depicts deployment of a set of electrodes 100 on and in a body, according to embodiments of the present invention. In this example, three pairs of surface electrodes (or surface pads) are shown: 102A/102B, 104A/104B and 106A/106B. The pairs of surface electrodes may be disposed on the body substantially at antipode locations. In some embodiments, a smaller or larger number of surface electrodes may be used, and their number may be even or odd. Additionally, set of electrodes 100 comprises intra-body electrodes 103. In the depicted embodiment, the intra-body electrodes are comprised in catheter 108. Catheter 108 may be insertable into a patient's body. In some embodiments, the intra-body electrodes may be carried by more than one catheter, for examples, two electrode-carrying catheters may be inserted into the patient's body, and used for generating an image as described below.

Surface electrodes 102A/102B, 104A/104B and 106A/106B may be connected to signal source(s) that is/are adapted to inject (or excite) electrical signals in desired strength, frequency and phase. In some embodiments the signal source may be tuned to excite each of the pairs of surface electrodes with signals having opposite phases (or at least substantially well de-phased from each other), for example in order to imitate three substantially spatially orthogonal axes (e.g. X, Y, and Z axes). This is so because such pairwise transmission may be used for locating a catheter, such as catheter 108, inside the body, e.g., for navigation purposes. In addition, voltages developing on the surface electrodes during the excitation of at least one of the intra-body electrodes may be measured and used (together with the known injected currents) for reconstructing a distribution of conductivity (or resistivity) in the volume defined by the body-surface electrodes, e.g., 3D conductivity map. This conductivity distribution may then be used for producing a 3D image of said volume.

Voltages developing on the surface electrodes and/or the intra-body electrodes during the excitation may be measured when the intra-body electrodes are actively moved (e.g., by a physician during a medical procedure) around a region of interest (or inside it or along it, etc.)—e.g., around or inside a tissue to be imaged. In some cases, there may be several regions of interest, and the intra-body electrodes may be “dragged” from one to another, back and forth. For example, inside a left atrium there are many structural features that may be of interest, e.g., the openings of the pulmonary veins (which are of high interest for treating atrial fibrillation), the left atrial appendage, the mitral valve, etc. The catheter may be guided to visit all of them (and especially those relevant to a current treatment), and so the image quality at these regions and their vicinity may be improved.

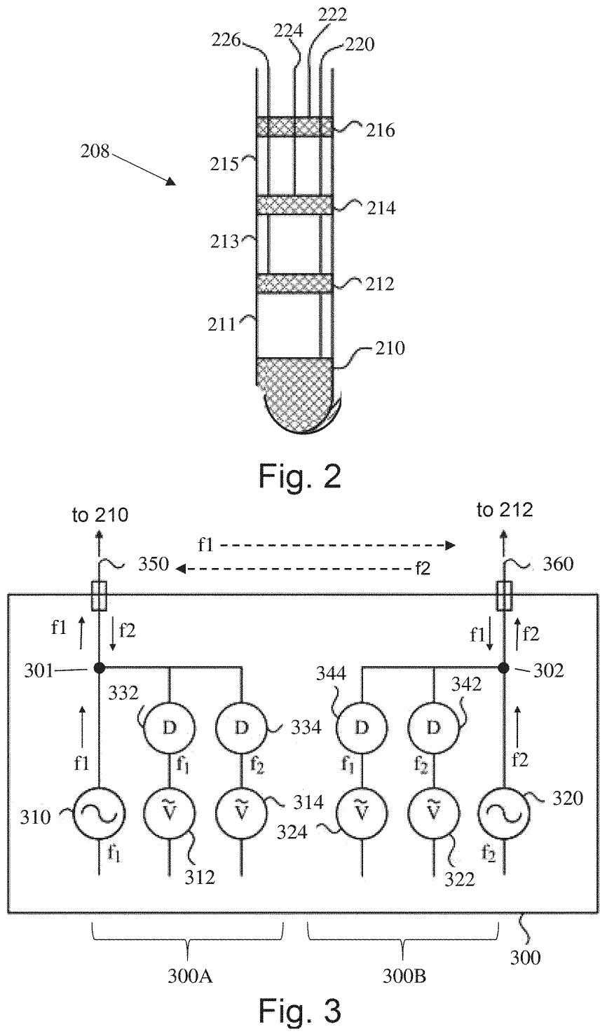

Reference is now made also to FIG. 2, which is a schematic illustration of catheter 208, according to embodiments of the present invention. Catheter 208 may be, in some embodiments, identical or substantially identical to catheter 108 of FIG. 1. Catheter 208 may comprise one or more electrodes (also referred to herein as intra-body electrodes or in-body electrodes), and in the drawn example four electrodes 210, 212, 214, 216. Each of the electrodes may have connection wire 220, 226, 224, 222, respectively, to enable connecting to electrical excitation unit, such as electrical field generator/measurer, e.g. as described with respect to FIG. 3 hereinafter. Electrodes 210, 212, 214, 216 may be disposed spaced from each other along the longitudinal axis of catheter 208 by longitudinal distances 211, 213, 215. The longitudinal distances may be, for example, in the range of lower than 1 millimeter or few millimeters and up to 1-2 cm or up to 4-6 cm between the farthest intra-body electrodes. In some embodiments it may be beneficial to have the electrodes spaced apart by a distance that is in the magnitude of order of the size of the scanned organ, or less.

In embodiments of the present invention schemes of electrical excitations of surface electrodes and/or catheter electrodes (also referred herein as excitation scheme or scheme of excitation) yield voltages measurable on one or more of the electrodes. The voltage readings (voltages measured on one or more surface electrodes and/or catheter electrodes) may be used to reconstruct a spatial distribution of the electrical conductivity of tissues through which the electrical signals pass (may be referred to herein as 3D conductivity map). Schemes of excitation may comprise selection of the transmitting electrode(s), selection of the frequency of the transmitted signals, selection of the amplitude of each of the transmitted signals, selected duration of the transmission, selection phase differences (or de-phasing) between signals transmitted concurrently from two or more electrodes at a same frequency, and the like. It will be noted that excitation schemes may comprise sets of signal frequencies (transmission frequencies) that may be selected to support one or more needs such as operating in different frequencies to cover different transmissivities of the body tissues along a certain signal path, thereby collecting more information of the tissue's shape. In another example, transmission frequencies may be selected to enable good separation between the transmitted and the received signal, or good separation between signals transmitted concurrently from different electrodes. While separating between signals transmitted concurrently from different electrodes may be achieved with signals separated from each other even in a few kHz, covering different transmissivities may benefit from large frequency differences, for example, frequencies spanning the frequency range between 10 kHz and 100 KHz.

Transmitted signals may be transmitted from one or more of the electrodes, and voltages developing on one or more of the electrodes during the excitation may be received and recorded for further processing. Preferably, voltages developing on all the electrodes are recorded. The voltages may be indicative of the conductivity of body tissues through which the signal passed. Since the conductivity along any electrical path of a signal is indicative of the nature of the tissue along that path, the more different signal paths are sampled, the richer is the data on the nature of the tissues, and a more accurate image (e.g., of higher resolution) may be produced from that data. Accordingly, excitation schemes may be used to invoke transmission from, for example, at least one of the catheter electrodes and the resulting voltages developing on at least all of the surface electrodes may be recorded, thereby providing, in the example of FIG. 1, indication of six different conductivities, which are indicative of the conductivity of the body tissues along six respective signal paths. The paths along which transmitted signals pass are not known, as the signals do not travel in straight lines, but mainly along paths of minimal resistivity. Yet, the large number of measurements of spatial conductivity values, which may represent, for a large number of points in the examined body organ, measurements of more than one signal path that passes through a certain point, enables reconstructing a detailed 3D map of conductivity values, which may be translated to a 3D image of the imaged tissue (e.g., of the organ).

In some embodiments, excitation schemes may be used to invoke transmission from at least one of the catheter electrodes and the resulting voltages developing on at least all of the intra-body electrodes may be recorded, thereby providing, in the example of FIG. 1, indication of four different signal paths, which are indicative of the conductivity of the body tissues along the respective paths.

Additionally, one or more transmitted signals may be transmitted from at least one of the surface electrodes and the resulting voltages developing on the other surface electrodes may be measured and recorded, thereby providing conductivity information related to signal paths through body tissues extending between the transmitting surface electrode and the at least one receiving surface electrode, which may provide indication of the tissues of the body closer to the body surface.

In some embodiments, at least some of the excitations may be by electrode pairs, transmitting simultaneously at the same frequency and in opposite phases. In some embodiments, such electrode pair may consist of two surface electrodes or two intra-body electrode electrodes. In some embodiments, such an electrode pair may consist of one intra-body electrode and one surface electrode.

In some embodiments, at least some of the excitations may be by electrode groups of three or more electrodes, transmitting simultaneously at the same frequency and in controlled phase relations between them. In some embodiments, each such electrode group may consist of intra-body electrodes or surface electrodes. In some embodiments, one or more of the groups may include both an intra-body electrode and a surface electrode.

In embodiments where the surface electrodes are also used for navigation, the surface electrodes may transmit in pairs (each pair transmitting at a common frequency, and in two opposite phases), and the intra-body electrodes may each transmit at a different frequency. In some such embodiments, voltages are read only on the intra-body electrodes, and in some embodiments, voltages are also read on the surface electrodes.

As mentioned above, processing of the measured voltages on the various electrodes may be used, additionally to the creation of database of 3D measurements (from which a 3D conductivity map may be produced, as is explained below), also for tracking and positioning the catheter inside the body. Tracking and positioning of the catheter inside the body may be used for medical procedures.

Adding catheter electrodes located inside the body to imaging systems (such as EIT systems) that use only body surface electrodes provides imaging data of much higher quality (e.g., of high definition), in comparison to what is achieved with imaging using only surface electrodes, at least with the same number of electrodes. The improved quality may be proved by comparing its resulting images to images of a known phantom of the body organ, directly measuring the conductivity at some points, and comparing the imaged values (i.e., the conductivity values obtained from the voltage readings) to the directly measured values. Alternatively to measuring a phantom, the quality may be evaluated by creating synthetic data (e.g., electromagnetic simulation), and comparing the imaging results to the simulation.

The plurality of voltage measurements v_{(i,j)} between pairs i, j of electrodes, performed as described above, when plurality of different excitations is applied along time to plurality of electrodes and measured by plurality of electrodes, creates a collection V_{(i,j)} of voltage measurements. The collection V_{(i,j)} of voltage measurements may be obtained when the intra-body electrodes are located at different positions within the body (e.g., as the catheter moves inside an organ).

The collection of voltage measurements may be converted to a collection of spatial conductivity values σ_{(x,y,z)}, assigning a calculated conductivity value to points in a defined 3D volume. The points σ_{(x,y,z)}, with their assigned conductivity values may be included in a large collection (or a cloud) of spatial values, hereinafter denoted R.

It will appreciated that the body volume that may be imaged according to embodiments of the present invention may be defined as a body volume confined between/among a set of surface electrodes usable in the imaging process.

In practice, the intra-body electrodes are typically catheter electrodes, so they may move with the catheter inside the body, when the catheter is moved, e.g. along a body lumen or inside a heart chamber or other organ(s). Solving the 3D conductivity map (i.e. calculating the conductivity value for the collection of 3D points in the scanned volume of the body based on voltages measured at the surface of the imaged volume and inside it or around it) may not require knowledge of the position of the electrodes, (other than knowing which are at the surface and which are inside the body), but the solution depends on that location.

Consider a catheter with a single electrode. At each position of the catheter, the readings at the catheter electrode (and at the surface electrodes, in the frequency transmitted by the catheter electrode) are different, and so are the reconstructions obtained from them. These different reconstructions may be combined, for example, by assigning to each region a conductivity value, equal to an average of the conductivity values assigned to that region in each of the different reconstructions. As the individual reconstructions may vary in quality, the averaging may be weighted by the quality.

Additionally or alternatively, different readings from different places may be combined to provide a single reconstruction. Excitations excited at different times don't interact with each other, similarly to excitations made simultaneously at different frequencies. Therefore, excitations made at different times may be combined as if they were taken simultaneously but at different frequencies.

When a plurality of intra-body electrodes is used, different electrodes may be injected with currents of different frequencies (unless the two electrodes share a differential excitation), e.g., in order to avoid interference between different excitations.

Combining data gathered at different times and at the same frequencies is similar to data gathered simultaneously at different frequencies. This is enabled due to the fact that the intra-body electrode carrier may be moved though the body lumen(s) thereby providing sets of spatial-related conductivity measurements representing large sets of conductance routes, which in turn enables enriching the 3D map of conductivity measurements and, as a result, improvement of the 3D resolution of the resulting image. According to embodiments of the present invention conductivity data (e.g., collection of voltage measurements) may be collected at high rates, so the number of conductivity data sets that may be combined may be in the order of magnitude of hundreds, and even thousands, so as to enable producing data/images at a rate of, for example, 100 Hz.

The frequency difference may be sufficiently small so that frequency-independent conductivity may be assumed. For example, in some embodiments, the frequency difference between two frequencies that may be injected simultaneously is 0.1 kHz. The entire frequency change is up to 150% of the lowest frequency. Frequencies of between 10 kHz and 100 kHz may be used. It will be apparent that other center—frequencies and other ranges of frequency deviations may be used, for example in order to collect data related to conductivity of body tissues at other frequencies. According to some embodiments, for a frequency-independent conductivity according to a reasonable approximation, small frequency differences (for example, from 10 kHz to 15 kHz in 0.1 kHz jumps,), may be used. For the collection of data also on conductivity at other frequencies, a 100 kHz frequency may be used, between 100 and 105 kHz.

Each electrode may measure voltages at each frequency, and it is assumed that the measurement is frequency independent. For example, if there are four intra-body electrodes, each of the intra-body electrodes is transmitting at a different frequency, and three pairs of surface electrodes, each pair transmitting at a different frequency, than each of the 10 electrodes may simultaneously measure voltages at 7 different frequencies.

When the catheter includes a plurality of electrodes, each time the catheter moves, the electrodes provide a different set of readings, and each such set may be reconstructed into a different conductivity image. The images can then be combined, e.g., by weighted averaging.

Alternatively, the readings may be combined into a single result set. For example, in the above-example of four intra-body electrodes and three pairs of surface electrodes, each position may provide seven readings at each of the 10 electrode, resulting a total of seventy (70) readings. Accordingly, N positions of the catheter may provide 70N readings that may be treated as if they were taken at 7N frequencies. Different frequencies that are used to imitate different positions do not interact with each other, so the total number of readings is 70 N. This single data set of 70 N readings may be processed to provide a single reconstruction of the imaged volume.

The conductivity distribution may be indicative of the anatomy, as different tissues have different conductivities, and even blood may have different conductivities depending on the oxygen concentration in it, so it is expected that, for example, the blood pool in the left atrium will show different conductivities at different distances from the ostium (openings) of the pulmonary veins. An anatomical image of a tissue (or a body organ) may be obtained from the conductivity distribution.

Reference is made now to FIG. 3 which schematically depicts electrical field generator/measurer 300, according to embodiments of the present invention. Field generator/measurer 300 of FIG. 3 depicts how two electrodes may be configured to transmit each at a different frequency, and receive (and measure) at this frequency, and at the frequency transmitted by the other electrode. Signal source 310 provides signal in frequency f1. This signal is fed to electrode, e.g., electrode 210 (of FIG. 2) via terminal point 350 and the signal reaches another electrode, e.g., electrode 212 (of FIG. 2) and received by it. Similarly, signal source 320 provides signal in frequency f2. This signal is fed to electrode 212 via terminal point 360 and the signal reaches electrode 210 and received by it. As a result, junction points 301 and 302 experience a multiplexed signal comprised of frequencies f1 and f2. D is a demultiplexer that is configured to receive, in the current example, multiplexed signal (comprising signals in frequencies f1 and f2) and enable only signal in one of the frequencies to pass through—signal in frequency f1 passes via D 332 and D 344 and signal in frequency f2 passes via D334 and D 342. Accordingly, voltmeter 312 measures the amplitude of the signal in frequency f1, as originated from signal source 310 and received by electrode 210, and voltmeter 314 measures the amplitude of signal in frequency f2 as originated from signal source 320 and received by electrode 210. The demultiplexing of the signals at section 300B of electrical field generator/measurer 300 is done in the same manner. Accordingly, voltmeter 324 measures the amplitude of the signal in frequency f1, as originated from signal source 310 and received by electrode 212, and voltmeter 322 measures the amplitude of signal in frequency f2 as originated from signal source 320 and received by electrode 212.

It will be apparent that for exciting more electrodes the sections 300A, 300B of electrical field generator/measurer 300 may be repeated. In some embodiments, other signal demultiplexers may be used, as is known in the art.

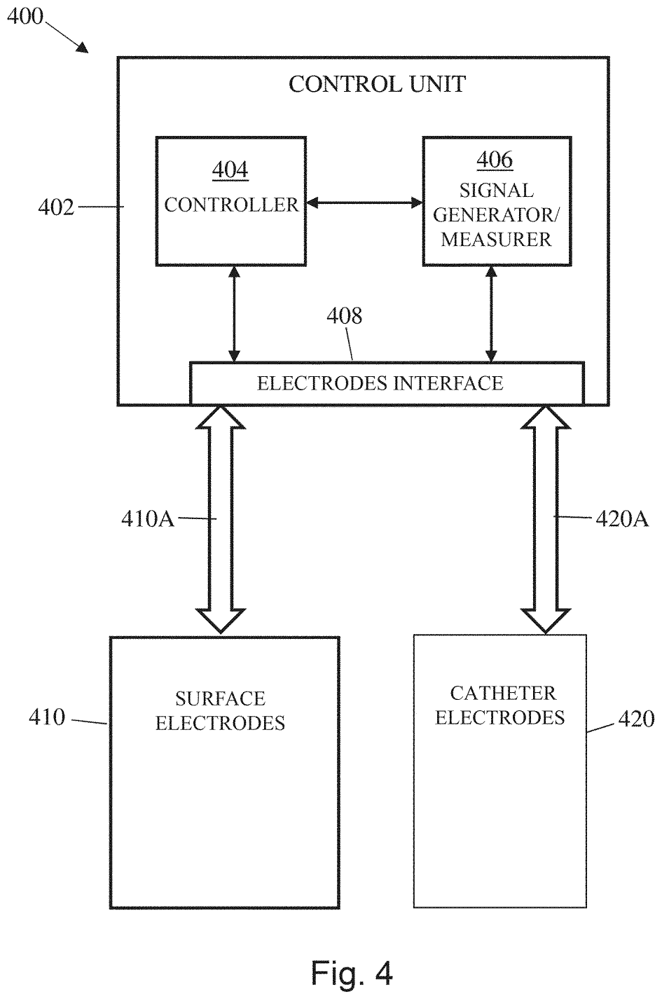

Reference is made to FIG. 4, which is a schematic block diagram of system 400 for conductivity-based imaging, according to embodiments of the present invention. System 400 may comprise main control unit 402 in active communication with surface electrodes unit 410 and intra-body electrodes unit 420, via communication channels 410A and 420A1 respectively. Main control unit 402 may comprise controller 404 and signal generator/measurer 406, connectable via electrodes I/O interface unit 408. Control unit 402 may include a controller that may be, for example, a central processing unit processor (CPU), a chip or any suitable computing or computational device, equipped with an operating system, a memory, an executable code, and a storage (not shown in order to not obscure the drawing). Main control unit 402 may be configured to carry out methods described herein, and/or to execute or act as the various modules, units, etc. More than one computing device may be included in a system according to embodiments of the invention, and one or more computing devices may act as the various components of the system. For example, by executing the executable code stored in the memory, the controller may be configured to carry out a method of acquiring signals from the electrodes for the construction of 3D imaging according to embodiments of the invention.

Signal generator/measurer 406 may produce signals in a manner similar to the description of the signals produced and measured by generator/measurer 300 of FIG. 3. Accordingly, signals may be fed to, and/or received from any of the body surface electrodes of surface electrodes unit 410 and catheter electrodes of intra-body electrodes unit 420. Body surface electrodes of unit 410 may be deployed and operated similarly to electrodes 102A/102B, 104A/104B 106A/106B of FIG. 1. Intra-body electrodes of unit 420 may be arranged and operable similar to electrodes 210, 212, 214 and 216 of FIG. 2.

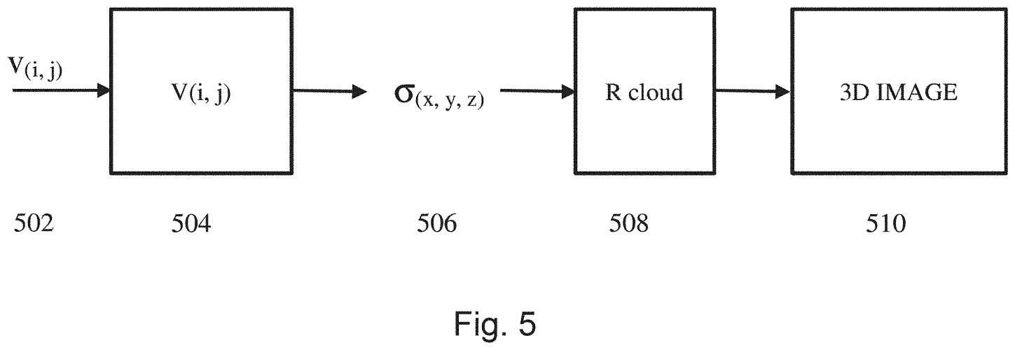

Reference is made to FIG. 5, which is a top-level flow of process 500 for converting a collection of measured voltages on a set of electrodes into a 3D image, according to embodiments of the present invention. Plurality of electrical signals may be injected to the electrodes, surface electrodes and catheter electrodes, according to one or more excitation schemes, as discussed above. A plurality of measured voltages v_{(i, j)} (502), measured at the plurality of electrodes, may be combined into a an averaged/weighted collection (or collections) V(i, j) (504) which then may be converted (or reconstructed) into large number of conductivity values σ_{(x, y, z)} (506), each is associated with a 3D point having a respective x, y, z spatial coordinates (508). The collection of spatial conductivity values may then be translated into a 3D image (510) that may be screened or otherwise presented.

Reference is made to FIG. 6A, which is a flow chart depicting method for conductivity-based imaging for imaging a body volume or for reconstructing body volume according to embodiments of the present invention. Body volume may include or be a body tissue. Currents may be injected, for example by control unit 402 using signal generator/measurer unit 406, to electrodes deployed on a patient's body, such as electrodes 410 of FIG. 4 (for example, electrodes 102A/B, 104A/B and 106A/B of FIG. 1), and to catheter electrodes, such as electrodes 420 of FIG. 4, for example electrodes 210, 212, 214 and 216 of FIG. 2, according to an injection scheme (block 602). Injection scheme may include a time/frequency transmit scheme. Injection scheme may be controlled and monitored by controller 404. At block 604, voltages are measured on electrodes (e.g., on all electrodes) e.g. by signal generator/measurer 406, and an inverse problem (calculation and production of 3D imaging of conductance of body tissues based on the currents/voltages inverse calculations) (block 606) may be solved, e.g. by control unit 402, and a 3D conductance map (3D distribution of conductance measurements, also referred to herein as conductivity map) may be obtained and optionally provided for display (block 608). At block 610, a 3D image of the body tissue may optionally be produced (and optionally presented) based on the 3D conductance map.

The same general scheme is applied also according to some embodiments of the present invention, as depicted in FIG. 6B, to which reference is now made. FIG. 6B is a flow diagram depicting method for performing multiple cycles of measurements, according to the invention. According to some embodiments, 1^{st} set of electrodes are attached (or deployed) on a patient's body (block 652) and 2^{nd} set of electrodes are inserted in a patient's body lumen (block 654). Currents are injected according to a scheme (block 656), the scheme may include a time/frequency transmit scheme. Voltages are measured on electrodes (e.g., all electrodes) (block 658), and an inverse problem (calculation and production of 3D imaging of conductance of body tissues based on the currents/voltages inverse calculations) (block 660) is solved and a 3D conductance map (3D distribution of conductance measurements) is provided (block 662). At block 664, a 3D image of the body tissue may optionally be produced (and optionally presented) based on the 3D conductance map. It will be noted that according to some embodiments, the measurement of voltages developing of a set of surface and intra-body electrodes may rely on electrodes already deployed at the patient's body (e.g., otherwise deployed as part of a medical procedure). In such conditions, use may be made of existing electrodes and the steps of blocks 652, 654 may not be required.

Reference is made to FIG. 7 which is a flow chart depicting a method of performing conductivity-based imaging according to some embodiments of the present invention. This flow chart depicts a method of collecting and combining sets of spatial data points representing conductance between sets of electrodes. The example of FIG. 7 includes a defined number (M) of repetitions of data collection, beginning with setting the iteration numerator to 1 at block 702. The sets of electrodes are excited according to a given excitation scheme (block 704) and the voltage developing on the electrodes are measured and recorded (block 706). At the end of each cycle of excitation and voltage reading the value of the cycle numerator is checked (block 708). In case it is lower than M, another cycle is performed (blocks 704, 706). When the numerator has reached the value of M, the recorded measurements are combined (block 710) for enhanced resolution of the extracted 3D conductance image. The combined measurements are used for solving the inverse problem (block 712). The solved inverse problem is used to providing a 3D conductance map of the measured tissue (block 714) and a 3D image may optionally be produced (and optionally presented) based on the 3D conductance map (block 716). The repetitions that are described above are of measurements made with the intra-body electrodes at different locations. The readings may be most sensitive to the immediate surrounding of the electrode, so reading at different locations may provide information on the conductivity in many different locations. The repetitions may preferably relate measurements taken at different locations of the intra-body electrodes. The number of iterations may be determined solely by the measurements rate multiplied by the time of performing of the medical procedure. In some other or additional embodiments the number of repetition of measurements may be determined by other limiter(s).

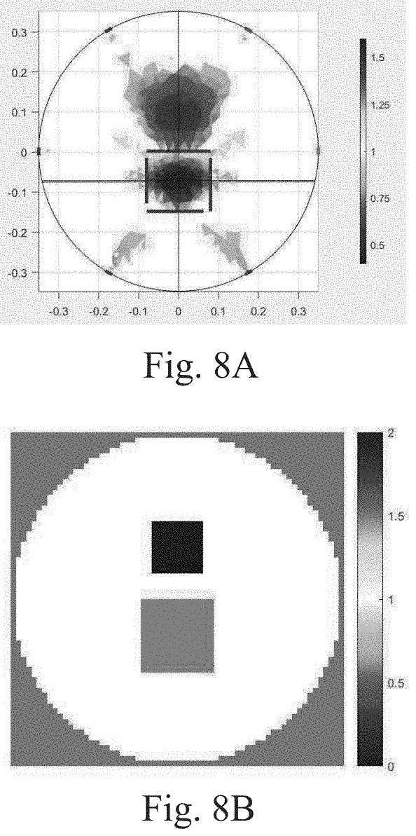

FIG. 8A, to which reference is now made, is a simulation results of 3D conductivity values reconstructed from voltage measurements made by surface electrodes and in-body electrodes according to some embodiments of the invention. The simulation was performed using a cylinder of conductivity 1.0 that included two bodies, one of conductivity 1.5, and the other of conductivity 0 (the latter may imitate an air column). FIG. 8B shows the actual structure the reconstruction of which is depicted in FIG. 8A. Electrodes are attached to the surface of the cylinder to simulate the surface electrodes, and to the higher conductivity object, to simulate the in-body electrodes. The Maxwell equations were solved for the configuration of FIG. 8B for excitations of signals of common frequency and opposite phase from the surface electrode, and excitations of signals of different frequencies by each of the in-body electrodes. Voltage readings at each of the electrodes were obtained from that solution. These voltage readings were inputted to a solution of the inverse problem, under the boundary conditions that intra-body electrodes are inside the cylinder, and the surface electrodes are at the outer surface of the cylinder. The inverse problem was solved using the MORS software, downloadable from www(dot)eidors3d(dot)sourceforge(dot)net/. As can be seen, the two objects are reconstructed with shape and conductivity values that are similar to those of FIG. 8B.

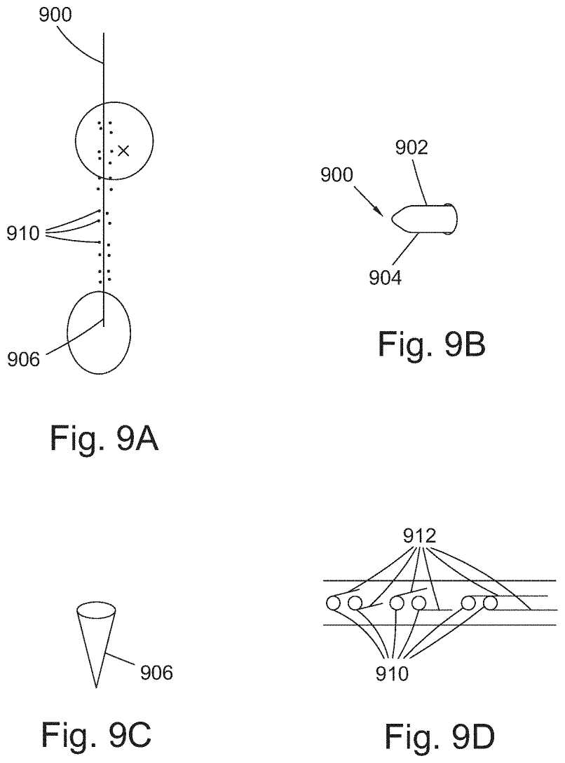

Reference is now made to FIGS. 9A-9D that illustrate a needle, according to some embodiments of the invention, which may be used for dielectric-based imaging. Needle may be an exemplary medical device used for the dielectric-based imaging.

FIG. 9A is a diagrammatic representation of a needle 900 according to some embodiments of the invention. Needle 900 may include two flat surfaces shown as surfaces 902 and 904 in FIG. 9B, which is a side view of needle 900. Each flat surface may have a width of about 0.7 mm and thickness of about 0.3 mm. The length of each of the flat surfaces is the length of needle 900, for example, between about 10 cm and about 20 cm. Needle 900 ends with a needle tip 906 at the distal end of the needle. FIG. 9C is an enlarged view of needle tip 906 according to some embodiments. Needle may be flexible or rigid.

Needle 900 may further include a plurality of electrodes 910 (referred as inner electrodes herein). The plurality of electrodes 910 may be arranged in pairs. In one example, the distance between the two electrodes in a pairs is about 2 mm (but may be in some embodiments between about 1 mm and about 5 mm), and the distance between two adjacent pairs is about 8 mm (but may be in some embodiments between about 4 mm and about 14 mm). In some embodiments, distance between adjacent electrodes is measured as the shortest distance between points on the circumference of the electrodes, (where each of the points is on a different one of the electrodes). Alternatively, distance between electrodes may be defined as a distance between centers of the electrodes. Distance between adjacent electrode pairs is the distance between two adjacent electrodes, each belonging to a different pair. Alternatively, distance between pairs (referred herein as inter-pair distance (D)) may be defined as a distance between the middle of each pair, wherein a middle of a pair is a middle of a line connecting the two centers of the electrodes making the pair. In the depicted embodiment, needle 900 includes 12 electrode pairs, 6 pairs on each flat surface. In some embodiments, the diameter of each electrode is about 0.6 mm. Here, and anywhere else in this disclosure, unless otherwise provided explicitly, the term “about” is to be understood to mean±10%. For example, about 0.6 mm means between 0.54 mm and 0.66 mm. FIG. 9D is a top view of needle 900, showing the flat surfaces 902 and 904, electrodes 910, and electrically conductive wires 912, each connecting a respective electrode to a measurement unit (e.g., signal generator/measurer 406) or a chip (not shown).

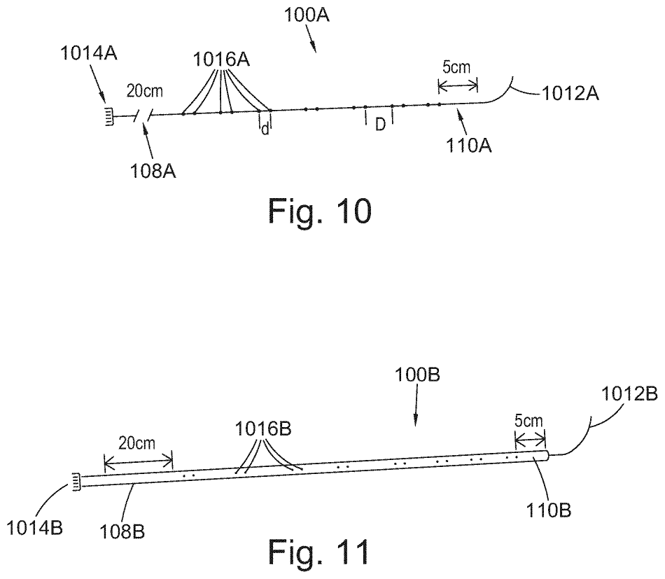

FIG. 10 is a diagrammatic illustration of a thread 100A, according to some embodiments of the invention, which may be used for dielectric-based imaging. Thread 100A may be an exemplary medical device used for the dielectric-based imaging. Thread 100A may be a suture thread. Suture thread may be made from textile material, for example: silk or another material—e.g., plastic material. Thread 100A is shown connected to a needle 1012A, which may be a suture needle, at the distal end of the thread. Needle 1012A may be according to the invention (e.g., as in FIG. 9A), or it may be conventional, i.e., without electrodes. At the proximal end thereof, suture thread 100A may have a connector 1014A, configured to connect the thread to a measurement device (e.g., signal generator/measurer 406). Thread 100A may include a plurality electrodes 1016A (referred as inner electrodes herein), optionally arranged in a plurality of electrode pairs. The distance between the two electrodes making the pair (d) is optionally about 2 mm, but may be in some embodiments between about 1 mm and about 5 mm.

It is noted that in some embodiments, solving the inverse problem may include applying constraints to the solution. The constraints may guide the solver towards solutions where features in the solution fit known features of the real world. For example, the constraint may guide the solver towards solutions where the distance between images of the electrodes, or points identified to have resistance similar to that of the electrodes, is equal to the known distance between the electrodes. Thus, the distance between the electrodes may affect the resolution of the image obtained. For example, in some embodiments, the closer are the electrodes to each other, the higher is the resolution. Therefore, it may be advantageous to have electrodes that are as close as possible to each other. In the example provided in FIG. 10, the difference d may be, for example, about 2 mm. The distance between the electrode pairs may be the same as the distance d, but then the number of electrodes may imply other limitation on the thread, for example, it may become heavy, less flexible, and not necessarily will improve the quality of the image to be obtained from readings of the electrodes. The inventor estimate that it may be most useful to use inter-pair distance (D) that is larger than the intra-pair distance (d) by a factor of between about 3 and about 5. For example, in some embodiments where d=2 mm, D=8 mm. Thus, the distance between the two most distant electrodes is, in this example, N cm, where N is the number of the electrode pairs. In the drawing, N=7, so the distance between the two furthest electrodes 1016A is 7 cm. In some embodiments, the inter-pair distance may vary along the thread, or along any other tool fabricated according to embodiments of the present invention. Similarly, intra-pair distances may also vary.

In some embodiments, each electrode in a pair faces a different direction, for example, one electrode may be at an upper surface of the thread and its pair may be at a lower surface of the thread. Thread 100A may include a “blind” part that does not include any electrode, or includes electrodes that do not take part in the dielectric imaging. For example, thread 1016A is shown to have a blind part 108A, between the electrodes 1016A and the connector 1014A. Blind part 108A may be about 20 cm in length. Thread 100A may also include blind part 110A at its distal end, where the thread connects to suture needles 1012A. Blind part 110A may be about 5 cm in length.

FIG. 11 is a diagrammatic presentation of a flex tape thread 100B with electrodes 1016B ((referred as inner electrodes herein) according to some embodiments of the invention. Thread 100B may be an exemplary medical device used for the dielectric-based imaging. Thread 100B is shown connected to connector 1014B at the proximal end, and to a needle 1012B at the distal end. Flex tape thread 100B may include a proximal blind portion 108B and a distal blind portion 110B. The figure shows one side of the tape, carrying 8 electrode pairs. The other side may have a similar number of electrodes, arranged in pairs in a similar manner, although in some embodiments the blind parts may be of different lengths (e.g., 20 cm and 5 cm on one side and 19.5 cm and 5.5 cm on the other side). In some embodiments, each side of flex tape thread includes 10, 12, 14, 16 or 18 pairs of electrodes.

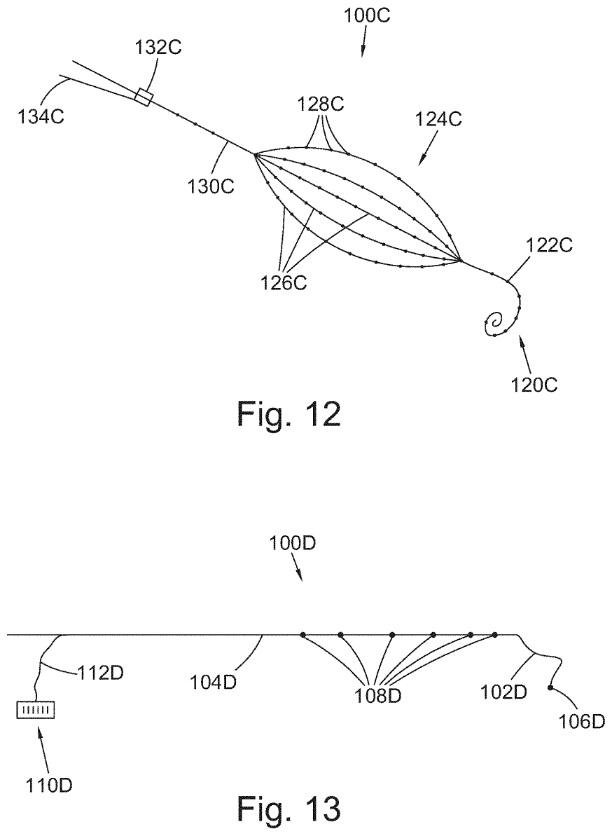

FIG. 12 is a diagrammatic presentation of a basket catheter 100C according to some embodiments of the invention. Basket catheter 100C may have a pigtail catheter portion 120C, with a plurality of inner electrodes 122C, optionally arranged in pairs, e.g., 3 or 4 electrode pairs. Basket catheter 100C further includes a basket portion 124C. The basket portion may comprise a plurality of strands 126C, for example, 8 strands or more, usually 12 strands or less, e.g., between 8 to 12 strands. Each strand 126C may include a plurality of inner electrodes 128C, optionally arranged in pairs.

Basket catheter 100C may further include a proximal catheter portion 130C. In some embodiments, proximal catheter portion is blind, i.e., with no electrodes. In some embodiments, proximal catheter portion 130C may include one or more inner electrodes, for example, 3 electrodes.

Basket catheter electrode 100C may include a chip 132C. The chip may receive conductive wires (not shown here, but see 912 in FIG. 9D) connecting the chip to each electrode of the basket catheter electrode 100C, including the electrodes at the proximal catheter portion, and the catheter portion (128C) and at the pigtail catheter portion (122C).

Chip 132C may include a D2A device, transforming digital data to analog signals. The D2A may be used to receive digital data through communication line 134C, and transferring them to analog signals, and transmit the analog signals to the electrodes. In some embodiments, the digital data includes a different set of instructions for each of the electrodes (or for different electrode groups), multiplexed so that each channel carries data with instructions to one of the electrodes. The chip may also include a demux, for demultiplexing the multiplexed signals received, and sending each set of instructions only to the electrode to which the instructions are addressed.

Chip 132C may include an A2D device, transforming analog signals to digital data. For example, to receive measurement results from the electrodes, and digitizing them, to send digitized measurement results through the communication line, for example, to a controller configured to receive the measurement results and analyze them (e.g., control unit 402 and/or controller 404). In some embodiments, some or all of the analysis is done at the chip, and the analysis results are sent via the communication line. Chip 132C may also include a multiplexer, for multiplexing digitized measurement results for sending via a single communication line 134C.

It should be noted that although chip 132C is disclosed in connection to FIG. 12, it may be included in any catheter or medical device described herein which may be used for dielectric-based imaging.

FIG. 13 is a diagrammatic presentation of a guidewire 100D according to some embodiments of the invention. Guidewire 100D has a flexible distal portion 102D connected to a rigid guidewire shaft 104D. The flexible distal portion 102D is optionally between about 1 cm long and about 6 cm long. At the distal end of distal portion 102D there is an electrode 106D, and the shaft includes 4, 6, 8, 10, or greater number of electrodes 108D (6 are shown). The distance between two adjacent electrodes 108D is from about 2 mm to about 12 mm, for example, about 6 mm.

Here, and in any of the other embodiment, e.g., of FIGS. 9A to 12, each electrode may be a ring electrode, having width of between about 0.2 mm and about 1 mm. The electrode may be made of gold, iridium, platinum, or an alloy of two or more thereof (e.g., 20% or 30% iridium, and the balance platinum). In some embodiments, tip electrode 106D is dome-shaped.

Shaft 104D is optionally connected to a connector, 110D, adapted to connect the guidewire to a to a measurement device (e.g., signal generator/measurer 406). Connector 110D may include a chip (not shown), similar to chip 132C, shown in FIG. 12.

The chip may receive conductive wires (not shown here, but see 912 in FIG. 9D) connecting the chip to each electrode of the guidewire 100D. The chip may include A2D device and D2A device, similarly to chip 132C. Connector 110D may be connected to guidewire shaft 104D via a flexible cable 112D.

Reference is made in this application to solving an inverse problem or obtaining a solution to an inverse problem. It will be understood that this refers to inferring a spatial distribution of conductance or another dielectric property based on the collection of measurements V(i,j) discussed above, that is based on measurements of voltages generated on (surface and/or in-body) electrodes in response to injection of currents to one or more (surface and/or in-body) electrodes. Finding the conductance distribution based on the measured voltages is the inverse of applying Maxwell or Laplace equations to currents applied at electrodes taking account of the conductance distribution and is hence referred to as solving an inverse problem. Many methods for doing this, numerical and otherwise will be known to the person skilled in the art and are available in commercially available software, for example as referenced above. One class of methods starts with a guess of a conductance distribution, applies the appropriate equations to the distribution and currents applied at one or more electrodes to produce predicted voltages at a set of electrodes at which voltages were measured. An error between the predicted and measured voltages is then used to adjust the conductance distribution and the process iterates until the error is reduced to a satisfactory level or another stopping criterion is met. In other words, this approach involves a form of optimization to find a spatial distribution of conductance that is consistent with the measured voltages in response to applied currents. One example of a class of methods of iteratively adjusting the conductance distribution in this way is gradient descent.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of performing dielectric-based imaging comprising:
exciting at least one pair of electrodes according to an excitation scheme, the at least one pair of electrodes comprising at least one pair of in-body electrodes located inside of an examined body or tissue;
measuring and recording voltages developing on the at least one pair of in-body electrodes during the excitation according to the excitation scheme, wherein the at least one pair of in-body electrodes is carried by a catheter or a guidewire actively moved in or around the examined body or tissue;
solving an inverse problem to obtain a 3D dielectric map from the recorded voltages; and
providing a 3D image of the body tissues based on the 3D dielectric map.

2. The method of claim 1, wherein the in-body electrodes are located inside a tissue of a living body.

3. The method of claim 1, wherein the at least one pair of in-body electrodes is carried by the catheter.

4. The method of claim 1, wherein the catheter is selected from the group consisting of: micro catheter, spiral catheter, basket catheter, pig tail catheter.

5. The method of claim 1, wherein the at least one pair of in-body electrodes is carried by the guidewire.

6. The method of claim 1, wherein the catheter comprises an inner tube and a sheath surrounding the inner tube, and wherein the at least one pair of in body electrodes is carried by the sheath.

7. The method of claim 1, wherein the at least one pair of in-body electrodes includes a plurality of pairs, each pair consisting of two electrodes distanced from each other by a distance of between 1 mm and 5 mm.

8. The method of claim 7, wherein the distance is between 1.8 mm and 2.2 mm.

9. The method of claim 7, wherein each two adjacent pairs of in-body electrodes consists of two pairs distanced from each other by a distance larger than the distance between electrode inside the pair by a factor of between 3 and 6.

10. The method of claim 9, wherein the factor is between 3.6 and 4.4.

11. A system for performing dielectric-based imaging comprising:
an excitation source, configured to electrically excite at least one pair of electrodes according to an excitation scheme;
wherein the excitation scheme provides for the excitement of the at least one pair of electrodes when comprising at least one pair of in-body electrodes located and moving inside of an examined body or tissue, and wherein the at least one pair of in-body electrodes is carried by a catheter or a guidewire;
a measuring and recording unit, configured to measure and record voltages developed on the at least one pair of in-body electrodes during the excitation according to the excitation scheme;
a processor configured to:
receive recorded voltages comprising voltages measured by the measuring and recording unit at different positions of the electrodes when the electrodes were actively moved in the region of interest,
solve an inverse problem to obtain a 3D dielectric map from the recorded voltages and the different positions of the electrodes during their measurement and
provide a 3D image of the body tissues based on the 3D dielectric map.

12. The system of claim 11, further comprising a catheter or a guidewire, the catheter or guidewire carrying the at least one pair of in-body electrodes.

13. The system of claim 11, wherein the catheter is selected from the group consisting of: micro catheter, spiral catheter, basket catheter, pig tail catheter.

14. The system of claim 11, wherein the at least one pair of in-body electrodes is carried by the guidewire.

15. The system of claim 11, wherein the catheter comprises an inner tube and a sheath surrounding the inner tube, and wherein the at least one pair of in body electrodes is carried by the sheath.

16. The system of claim 11, wherein the at least one pair of in-body electrodes includes a plurality of pairs, each pair consisting of two electrodes distanced from each other by a distance of between 1 mm and 5 mm.

17. The system of claim 16, wherein the distance is between 1.8 mm and 2.2 mm.

18. The system of claim 16, wherein each two adjacent pairs of in-body electrodes consists of two pairs distanced from each other by a distance larger than the distance between electrode inside the pair by a factor of between 3 and 6.

19. The system of claim 18, wherein the factor is between 3.6 and 4.4.

20. A guidewire comprising:
a guidewire shaft carrying a plurality of electrodes;
a distal flexible guidewire portion attached to a distal end of the guidewire shaft, and carrying a tip electrode at a distal end of the distal flexible guidewire portion, and
a proximal flexible cable, connecting the guidewire shaft to a connector adapted to connect to a measuring device, and comprising a chip including an A2D device, a D2A device, and conducting wires, conducting signals from the plurality of electrodes and/or from the tip electrode to the A2D device and to the D2A device.

21. The guidewire of claim 20, wherein each of the electrodes carried by the shaft is ring shaped.

22. The guidewire of claim 20, wherein the tip electrode is dome-shaped.

23. The guidewire of claim 20, wherein the electrodes are made of iridium, gold, platinum, or an alloy comprising two or more of iridium, gold, and platinum.

24. The method of claim 1, comprising repeating the exciting and the measuring, wherein the electrodes are actively moved between repetitions of the exciting.

25. The method of claim 1, comprising combining measurements obtained when the in-body electrodes were at different locations to a single set of measurements; and wherein solving the inverse problem comprises solving for that single set of measurements.

26. The system of claim 11, wherein the processor is configured to:
combine measurements obtained when the in-body electrodes were at different locations to a single set of measurements; and
solve the inverse problem for said single set of measurements.
